(19) United States
(12) Patent Application Publication
(10) Pub. No.: US 2021/0023321 A1
(43) Pub. Date: Jan. 28, 2021

(54) Bronchoendoscopic Intubation Guide Device

(71) Applicant: The 5137 Arrowhead Lakes Drive, LLC, Glendale, AZ (US)

(72) Inventor: Jugroop Brar, Glendale, AZ (US)

(73) Assignee: The 5137 Arrowhead Lakes Drive, LLC, Glendale, AZ (US)

(21) Appl. No.: 16/520,128

(22) Filed: Jul. 23, 2019

(51) Int. Cl. A61M 16/04

(52) U.S. Cl. CPC A61M 16/0493; A61M 16/0402; A61M 16/0497

(57) ABSTRACT

A device includes a bite block and a guide formed monolithically thereto and extending distally therefrom. The bite block includes a retention structure at a proximal end of the device and a stub. The stub extends behind the retention structure and has a bite area which is sized to closely receive a mouth. The bite block and guide are formed by a common sidewall, extending continuously from a proximal end, at the retention structure, to an opposed distal end of the device.

### FIELD OF THE INVENTION

The present invention relates generally to medical devices, and more particularly to endoscopic devices.

### BACKGROUND OF THE INVENTION

Oral intubation is often a necessary technique for establishing an airway in patients during surgery or other medical procedures. Oral intubation presents special challenges. For instance, the patient's tongue and soft tissues can fall backward and obstruct the airway during the procedure. Negotiating the pharynx requires making a sharp turn from the oral cavity down; some instruments can damage the pharynx if the medical worker cannot direct them through the pharynx properly.

Because of challenges such as these, medical workers often use a rigid guide that maintains the airway and directs an endoscope or other tools. However, often times, other apparatus need to be disposed in or around the mouth. For instance, for a patient in need an oxygen supply, a face mask can provide oxygen to the nose and mouth to facilitate normal breathing. The mask can be awkward for the patient and doctor, and use of a mask together with a rigid guide can be cumbersome if one must be moved or manipulated. The guide can prevent forming a good deal between the mask and the patient's face, and the mask also makes access to the guide difficult.

In some cases, the rigid guide may inadvertently move in the patient's mouth. This can present a choking hazard if the guide falls backward. It can also dislodge endotracheal equipment if the patient spits the guide forward. It can be knocked from position if the patient moves or is moved.

Challenges such as these are still unmet. A need exists for a way to intubate a patient, maintain the patient's airway, and minimize the movement and confusion of many devices around the patient's mouth and within the patient's pharynx.

### SUMMARY OF THE INVENTION

A device includes a bite block and a guide formed monolithically thereto and extending distally therefrom. The bite block includes a retention structure at a proximal end of the device and a stub. The stub extends behind the retention structure and has a bite area which is sized to closely receive a mouth. The bite block and guide are formed by a common sidewall, extending continuously from a proximal end, at the retention structure, to an opposed distal end of the device.

Another embodiment of a device includes a passageway bound by a sidewall extending from a proximal end of the device to a distal end of the device, the sidewall having a straight portion proximate the proximal end and extending linearly back from the proximal end, and a curved portion proximate the distal end and curving downward from the straight portion. Retention structure is at the proximal end of the sidewall and surrounds an aperture into the passageway. The sidewall extends continuously and monolithically from the retention structure to the distal end of the passageway.

Another embodiment includes a bite block and a guide formed monolithically thereto and extending distally therefrom, each bounding and defining a passageway. The bite block includes a retention structure at a proximal end of the device, and a stub extending behind the retention structure. The stub has a bite area which is sized to closely receive a mouth. The guide curves downwardly from the bite block and has an open bottom. The bite block and guide are formed by a common sidewall, extending continuously from a proximal end, at the retention structure, to an opposed distal end of the device.

The above provides the reader with a very brief summary of some embodiments discussed below. Simplifications and omissions are made, and the summary is not intended to limit or define in any way the scope of the invention or key aspects thereof. Rather, this brief summary merely introduces the reader to some aspects of the invention in preparation for the detailed description that follows.

### BRIEF DESCRIPTION OF THE DRAWINGS

Referring to the drawings:

FIGS. 1 and 2 are front and rear perspective views, respectively, of an embodiment of a bronchoendoscopic intubation guide device;

FIG. 3 is a front elevation view of the device of FIG. 1;

FIG. 4 is a section view of the device of FIG. 1 taken along the line 4-4 in FIG. 1; and

FIG. 5 is a partial section view showing the device of FIG. 1 in use with a patient.

### DETAILED DESCRIPTION

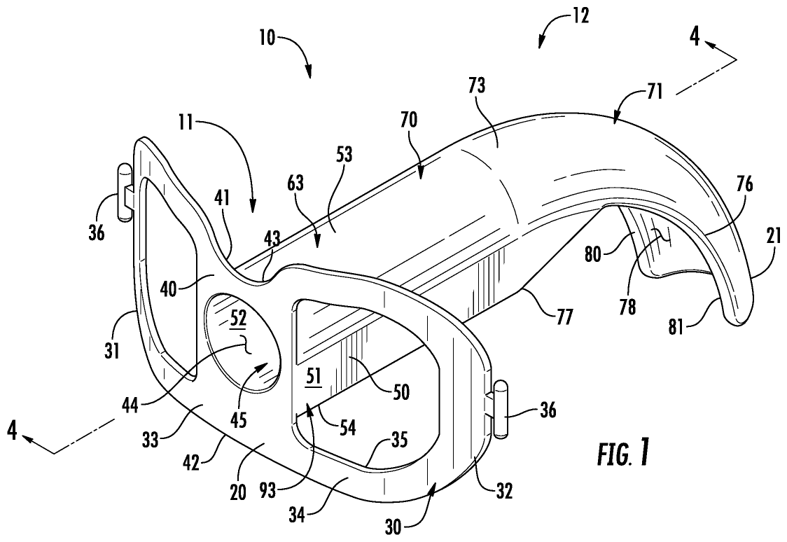

Reference now is made to the drawings, in which the same reference characters are used throughout the different figures to designate the same elements. FIG. 1 illustrates an integrated bronchoendoscopic intubation guide device (hereinafter, the “device” 10). The device 10 combines a bite block 11 with a guide 12 to provide an apparatus which health workers can insert into an operative patient's mouth where it will be held securely and reliably while establishing and maintaining an airway and an access into the patient's pharynx.

The device 10 has a proximal end 20 and an opposed distal end 21. The bite block 11 is located proximate to the proximal end 20, and the guide 12 extends back to the distal end 21. Generally, terms like “proximal,” “front,” “ahead,” and the like are used to define the location of an element of feature, or the relative arrangement of elements or features with respect to each other, and are meant to indicate a direction toward the proximal end 20. Similarly, terms like “distal,” “back,” “behind,” and the like are used to define the location of an element of feature, or the relative arrangement of elements or features with respect to each other, and are meant to indicate a direction toward the distal end 21.

The bite block 11 has a forward facial retention structure 30, a stub 50 projecting distally from the retention structure 30, and a bite area 63 on the stub 50. The guide 12 extends distally from the bite area 63.

The facial retention structure 30 is at the proximal end 20 of the device 10. The retention structure 30 includes two wings 31 and 32 extending laterally from the sidewall of the device 10 and oppositely to each other from a center portion 33. The wings 31 and 32 are thin, arcuate U-shaped elements. They are flat in cross-section and have opposed front and back sides 34 and 35. The front and back sides 34 and 35 are flat. The wings 31 and 32 are each formed with a distal bend so that together they form a convex shape applicable over the mouth and surrounding area of a patient. At opposed sides of the wings 31 and 32 are small cleats 36 to which opposed ends of an elastic band or strap can be secured. The band is wrapped around the patient's head to hold the device 10 to the patient.

The center portion 33 is located between the opposed wings 31 and 32. The center portion 33 has a flat front face 40 and an opposed flat rear face 41. The wings 31 and 32 are extensions of the center portion 33, and indeed, a bottom 42 and top 43 of the center portion 33 are contiguous to the wings 31 and 32. The bottom 42 is roughly linear, while the top 43 is notched inward to accommodate the lower end of a patient's nose when the device 10 is in use. An aperture 44 is formed near the middle of the front face 40, the aperture 44 opening into a roughly cylindrical passageway 45 within the device 10.

The bite block 11 further includes the stub 50 distal to the retention structure 30. The stub 50 is a short projection extending from the back of the retention structure 30 in a direction normal to the rear face 41 of the center portion 33. The stub 50 has an outer surface 51 and an opposed inner surface 52. The outer surface 51 extends rearwardly from the rear face 41 and is contiguous to an outer surface of the guide 12, while the inner surface 52 extends from the aperture 44 and is contiguous to an inner surface of the guide 12. The outer surface 51 has a semi-rectangular cross-section or profile with a curved or semi-annular top 53, a flat bottom 54, and opposed flat sides 55 and 56. The top 53 and bottom 54 are parallel and each perpendicular to the parallel sides 55 and 56. The inner surface 52 is round, defining a proximal portion of the passageway 45.

The stub 50 terminates distally at the guide 12, which defines the distal end of the bite block 11. A bite area 63 is disposed between the retention structure 30 at a proximal end of the bite area 63, and the guide 12 at a distal end of the bite area 63. The bite area 63 covers the stub 50 and includes the top 53 and bottom 54 of the outer surface 51 thereof. This bite area 63 is sized to closely receive the mouth—the lips, teeth, and oral cavity above the oropharynx—of a patient. The distance between the retention structure 30 and the guide 12 is approximately forty to eighty millimeters, which is approximately the width of a patient's oral cavity. In other embodiments, of course, differently-sized bite areas 63 accommodate differently-sized mouths. As such, the patient's lips can wrap around the stub 50, and the teeth can bite down against the bite area 63, so that the mouth is closely received behind the retention structure 30, over the bite area 63.

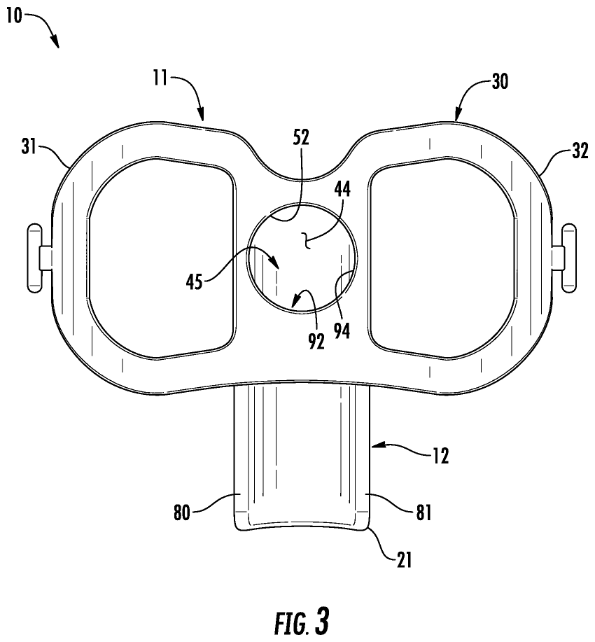

The bite block 11 and the guide 12 are integrally and monolithically formed to each other. Indeed, the device 10 is preferably formed from a single piece, constructed from a material or combination of materials having rigidity, such as high-density plastic. The device 10 has a body formed by a single, common, continuous sidewall 13, as shown in FIG. 4. This sidewall 13 has extrusions or projections, such as the retention structure 30, but generally, the device 10 is defined by this sidewall 13. As can be seen in FIG. 4, the sidewall 13 continues without discontinuity or interruption from the proximal end 20 to the distal end 21.

Referring now to FIGS. 1, 2, and 4, the sidewall 13 extends behind the bite area 63 of the bite block 11 to form the guide 12. The sidewall 13 has a straight portion 70, defined by the bite block 11, and a curved portion 71, defined by the guide 12. The straight portion 70 extends contiguously, linearly, and distally in alignment with and from the stub 50. Along the straight portion 70, the bite block 11 is enclosed. The guide 12, however, is opened. The guide 12 has a top 73 and opposed sides 75 and 76. The top 73 and sides 75 and 76 cooperate to partially enclose the passageway 45, which continues without interruption from the opening 44, through the stub 50, and into the guide 12. The top 73 of the guide 12 is an extension of the top 53 of the stub 50, and the sides 75 and 76 are extensions of the sides 55 and 56, respectively.

The bottom 54 of the stub 50 terminates at a distal edge 77 of the straight portion 70. The distal edge 77 is a flat edge extending between the opposed sides 75 and 76. The top 73 and opposed sides 75 and 76 continue along the curved portion 71. The straight portion 70 of the sidewall 13 is straight both along the bite block 11 and behind the bite block 11 up to the distal edge 77; it projects directly back from the retention structure 30, normal thereto, and continues in a consistent direction until the curved portion 71. The curved portion 71 then curves downward behind the straight portion 70. The curved portion 71 makes an approximately eighty-five degree bend, such that, at the distal end 21, the top 73 is oriented nearly perpendicularly to its alignment along the straight portion 73. Because there is no closed bottom along the curved portion 71, the sides 75 and 76 have lower edges 80 and 81, respectively. These lower edges 80 and 81 extend from the distal edge 77 at the end of the straight portion 70 to the distal end 21 of the device 10. They are opposed from each other and define an open bottom 78 to the curved portion.

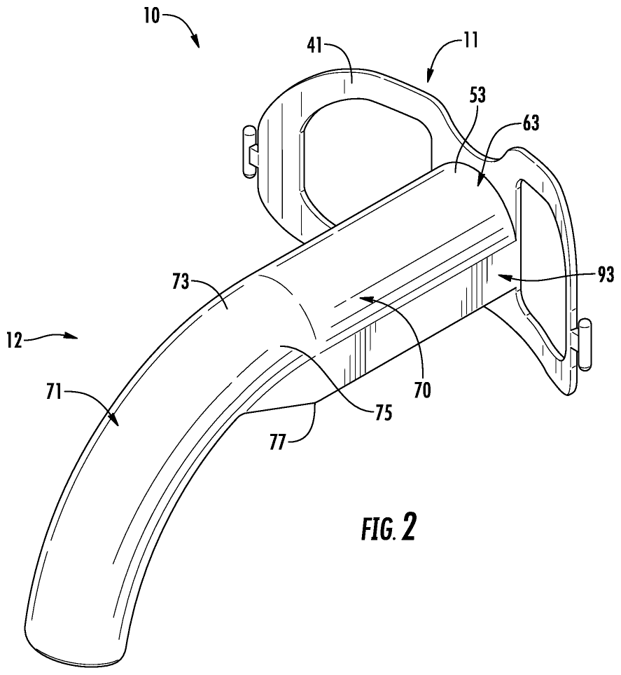

The sidewall 13 further has an inner profile 92 and an opposed outer profile 93. The inner profile 92 is best shown in FIG. 3, and the outer profile 93 is best shown in FIGS. 1 and 2. The inner profile 92 is defined by an inner surface 94 of the sidewall 13 which extends continuously from the aperture 44 to the distal end 21 of the device 10. This inner surface 94 is contiguous to the inner surface 52 of the stub 50. The inner profile 92 is round. From the aperture 44 at the proximal end 20 to the distal end of the straight portion 70, the inner profile 92 is annular, having an upper semi-annular or semi-cylindrical profile and an opposed lower semi-annular or semi-cylindrical profile between which there are preferably no seams, discontinuities, or other irregularities, though other embodiments may have such features. At the distal end of the straight portion 70, the sidewall 13 continues distally only from the top 73 and the sides 75 and 76. Just past the distal edge 77, the sides 75 and 76 transition obliquely up from the distal edge 77 to the lower edges 80 and 81. From the proximal end of the lower edges 80 and 81 to the distal end 21 of the device 10, the inner profile 92 is semi-annular, because only the upper semi-annular profile continues beyond the straight portion 70. Therefore, while the inner profile 92 is annular along the straight portion 70, it is semi-annular along the curved portion 71. There are preferably no seams, discontinuities, or other irregularities in the inner profile 92 such that the inner profile 92 is smooth, though other embodiments may have such features. Indeed, preferably, the entire inner profile 92 is smooth.

The outer profile 93 of the sidewall 13 also changes shape along the length of the device 10. The outer profile 93 is defined by an outer surface 95 of the sidewall 13 which extends from the retention structure 30, over the bite area 63, and to the distal end 21 of the device 10. Along the bite area 63, the outer profile 93 is semi-rectangular and semi-cylindrical, as seen in FIGS. 1 and 2. The lower half of the outer profile 93 has this rectangular profile until the distal edge 77, where it terminates. Along that same length, and continuing on entirely to the distal end 21 of the device 10, an upper half of the outer profile 92 is semi-annular or semi-cylindrical. Therefore, along the straight portion 70, the outer profile 92 is semi-rectangular and semi-annular, while along the curved portion 71, both the inner and outer profiles 92 and 93 of the guide 12 are semi-annular, since the bottom 78 is open. There are preferably no seams, discontinuities, or other irregularities in the outer profile 93 such that the outer profile 93 is smooth, though other embodiments may have such features. Indeed, preferably, the entire outer profile 93 is smooth.

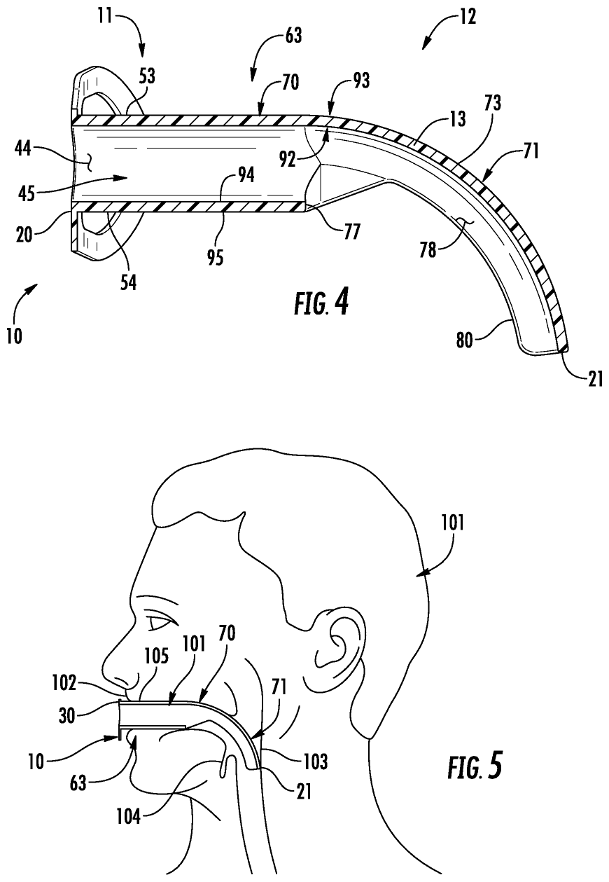

Referring now to FIG. 5, in operation, the device 10 is placed into a mouth 100 of a patient 101 and inserted until the retention structure 30 is against the front of the patient's lips 102. Generally, this will locate the distal end 21 of the device 10 in the pharynx 103 proximate the epiglottis 104. The straight portion 70 of the device 10 is disposed within the oral cavity, and the curved portion 71 is disposed within the pharynx, oriented downward and aligned with that passageway.

The mouth 100 of the patient 101, who is generally anesthetized, is manipulated so that the patient's lips 102 and teeth 105 are closely received in the bite area 63. By closing the patient's mouth 100, the device 10 is held; rearward movement of the device 10 is prevented by interaction of the lips 102 against the retention structure 30. The device 10 may be further secured and prevented from forward movement by applying an elastic band on the cleats 36 on the opposed wings 31 and 32, as described above, and wrapping the band around the patient's head. With the device 10 so secured, medical workers can apply instruments and tools through the aperture 44 and into the passageway 45 to guide the instruments and tools into the patient's body, while maintaining the passageway 45 as an air pathway. Because the guide 12 and bite block 11 are integrally and monolithically formed, the guide 12 can be moved within the patient's pharynx 103 by moving the bite block 11 or even just by repositioning the retention structure 30.

A preferred embodiment is fully and clearly described above so as to enable one having skill in the art to understand, make, and use the same. Those skilled in the art will recognize that modifications may be made to the description above without departing from the spirit of the invention, and that some embodiments include only those elements and features described, or a subset thereof. To the extent that modifications do not depart from the spirit of the invention, they are intended to be included within the scope thereof.

The invention claimed is:

1. A device comprising:
a bite block and a guide formed monolithically thereto and extending distally therefrom;
the bite block comprises:
a retention structure at a proximal end of the device; and
a stub extending behind the retention structure, wherein the stub has a bite area which is sized to closely receive a mouth;
wherein the bite block and guide are formed by a common sidewall, extending continuously from a proximal end, at the retention structure, to an opposed distal end of the device.

2. The device of claim 1, wherein:
the sidewall has a straight portion, which is straight both along and behind the bite block; and
the sidewall has a curved portion, which curves downward behind the straight portion.

3. The device of claim 2, wherein:
the sidewall has an inner profile and an opposed outer profile;
along the straight portion, the inner profile is annular and the outer profile is semi rectangular and semi-annular; and
along the curved portion, the inner and outer profiles are both semi-annular.

4. The device of claim 2, wherein:
along the straight portion, the guide is enclosed; and
along the curved portion, the guide has an open bottom.

5. The device of claim 1, wherein the retention structure includes opposed wings extending laterally from an aperture into the guide.

6. The device of claim 1, wherein the inner profile is smooth and without discontinuities.

7. A device comprising:
a passageway bound by a sidewall extending from a proximal end of the device to a distal end of the device, the sidewall having a straight portion proximate the proximal end and extending linearly back from the proximal end, and a curved portion proximate the distal end and curving downward from the straight portion;
retention structure at the proximal end of the sidewall, the retention structure surrounding an aperture into the passageway; and
the sidewall extends continuously and monolithically from the retention structure to the distal end of the passageway.

8. The device of claim 7, wherein the retention structure includes opposed wings extending laterally from the aperture.

9. The device of claim 8, wherein the retention structure extends laterally from the sidewall.

10. The device of claim 7, wherein:
the sidewall has an inner profile and an opposed outer profile;
along the straight portion, the inner profile is annular and the outer profile is semi-rectangular and semi-annular; and
along the curved portion, the inner and outer profiles are both semi-annular.

11. The device of claim 7, wherein the inner profile is smooth and without discontinuities.

12. The device of claim 7, wherein along the curved portion of the passageway curving downward from the enclosed portion, the passageway has an open bottom.

13. A device comprising:
a bite block and a guide formed monolithically thereto and extending distally therefrom, each bounding and defining a passageway;
the bite block comprises a retention structure at a proximal end of the device, and a stub extending behind the retention structure, wherein the stub has a bite area which is sized to closely receive a mouth; and
the guide curves downwardly from the bite block and has an open bottom;
wherein the bite block and guide are formed by a common sidewall, extending continuously from a proximal end, at the retention structure, to an opposed distal end of the device.

14. The device of claim 13, wherein the retention structure includes opposed wings extending laterally from an aperture leading to the passageway at the proximal end of the sidewall.

15. The device of claim 14, wherein the retention structure extends laterally from the sidewall.

16. The device of claim 13, wherein:
the sidewall has an inner profile and an opposed outer profile;
along the bite block, the inner profile is annular and the outer profile is semi-rectangular and semi-annular; and
along the guide, the inner and outer profiles are both semi-annular.

17. The device of claim 16, wherein the inner profile is smooth and without discontinuities.

18. The device of claim 13, wherein the bite block is enclosed by the sidewall.
